(19) United States
(12) Patent Application Publication
(10) Pub. No.: US 2022/0260576 A1
(43) Pub. Date: Aug. 18, 2022

(54) CANCER STATUS PREDICTION METHOD AND USES THEREOF

(71) Applicant: Chang Gung University, Taoyuan (TW)

(72) Inventors: Min-Hsien Wu, Taoyuan (TW); Po-Yu Chu, Taoyuan (TW)

(21) Appl. No.: 17/228,921

(22) Filed: Apr. 13, 2021

(30) Foreign Application Priority Data
Feb. 17, 2021 (TW) 110105324

(51) Int. Cl. G01N 33/574; G16H 50/20; G16H 10/40

(52) U.S. Cl. CPC G01N 33/57492; G16H 10/40; G16H 50/20

(57) ABSTRACT

A cancer status prediction method and uses thereof, by analyzing specific cell information in blood samples and cancer clinical detection data with a cancer status analysis module to further perform cancer status prediction.

### REFERENCE TO RELATED APPLICATIONS

The present application is based on, and claims priority from, Taiwan application number 110105324, filed Feb. 17, 2020, the disclosure of which is hereby incorporated by reference herein in its entirety.

### BACKGROUND OF THE INVENTION

### 1. Field of the Invention

The present disclosure relates to the field of cancer sample analysis and disease condition prediction. Specifically, the present invention relates to a method for separating and analyzing specific nucleated cells in a blood sample, and combining the above analysis results with cancer clinical detection data to further predict the cancer state.

### 2. Description of the Prior Art

Currently, cancer detection is mainly divided into three categories: imaging diagnosis, tissue biopsy, and cancer blood detection. There are many methodologies for imaging diagnosis, such as computerized tomography (CT), positron emission tomography (PET), nuclear magnetic resonance imaging (NMRI), ultrasound imaging, etc. However, imaging diagnosis requires cancer tissue growing to a certain size (0.5˜1 cm) for clearly identified, and it is prone to discrepancies in judgment. Second, most imaging diagnoses use imaging agents and radiation exposure which tends to accumulate damage to the subject's body by the increase in the number of diagnoses. Therefore, it cannot be used multiple times in a short time so that it is difficult to achieve the purpose of real-time monitoring of the cancer development of patients. On the other hand, tissue biopsy technology requires surgery or puncture the subject to obtain cancer tissue in the body. The sampling method is highly invasive and harmful and cannot be used as a long-term cancer monitoring method.

Unlike the above two methods, cancer blood detection only requires blood sampling to obtain cancer-related information. Therefore, its low-invasive and low-damage detection process is suitable as a long-term and real-time cancer monitoring and detecting method. Most of the current cancer blood detection commonly used by medical therapy unit is to measure the concentration of specific tumor markers in the blood samples of the subjects, that is, to measure and analyze the concentration of specific protein targets secreted by cancer cells to evaluate and monitor the cancer status, treatment effect or tracking the cancer recurrence in patients. However, the detection value may be affected by other physiological factors (such as inflammation, smoking, etc.), and the sensitivity and specificity of cancer are too low to accurately reflect the current status of cancer.

Different from the limitation of detection sensitivity of traditional cancer blood detection methods (i.e., blood tumor markers), circulating tumor cells, as an emerging cancer detection indicator, are formed by a group of cancer cells that detach from the primary tumor site and enter the blood circulation. The number of the cancer cells in the blood is highly correlated with cancer metastasis. Many studies have shown that the detection of the number of circulating tumor cells in a fixed blood volume can be used to evaluate the prognosis of cancer patients, to evaluate the effectiveness of anticancer drug treatment, or to track the recurrence of cancer after surgery. Moreover, the specificity related to cancer status is far superior to traditional hematological tumor markers. In the inventor's prior research, in addition to verifying the number of circulating tumor cells in blood samples of different types of cancer patients is higher than that of healthy subjects, a group of specific cells that are highly related to the development of cancer, nucleated cells with negative tumor surface markers and negative blood cell surface markers, were discovered (refer to the Republic of China Invention Patent Application No. 108114158). However, it is theoretically believed that circulating tumor cell information is helpful for the clinical detection of cancer. In practice, the number of circulating tumor cells in blood samples is relatively rare, thus affecting the assessment of cancer state. Meanwhile,

restricted in the difficulty to distinguish the origin of the tumor in situ, the clinical application of current detection methods of circulating tumor cells limited to cancer development monitoring and treatment effectiveness evaluation in cancer patients. On the other hand, most studies generally believe that only relying on the number of circulating tumor cells cannot accurately distinguish different cancer stages or distinguish metastatic and non-metastatic cancer, which further limits the possibility of circulating tumor cells in clinical detection.

As mentioned previously, there is an urgent need for a cancer status prediction method that not only has low invasiveness and low harm, but also has high specificity and sensitivity, and can be used to predict the status of metastatic/non-metastatic cancer and early-stage/late-stage cancer.

### SUMMARY OF THE INVENTION

In view of this, in addition to purifying, analyzing and measuring specific cells in the blood sample, the inventor also combined a cancer detection data for common analysis; thereby, not only improved the specificity and sensitivity but also predicted the status of metastatic/non-metastatic cancer and early-stage/late-stage cancer.

The present invention offers a method for cancer status prediction and uses thereof, which is helpful for accurately predicting cancer status such as early-stage/late-stage cancer, metastatic/non-metastatic cancer under the conditions of low invasiveness and low harm. The present disclosure further achieve the purpose of diagnosing cancer, evaluating cancer prognosis, tracking and monitoring cancer or evaluating the effectiveness of cancer treatment.

One aspect of the present invention is to offer a method for predicting cancer status, comprising the following steps:

Step 1: providing a whole blood sample from an individual.

Step 2: performing a process on the whole blood sample to remove plural erythrocytes and platelets to further obtain a processed sample.

Step 3: screening the processed sample by screening out at least one blood cell whose blood cell surface is marked as positive, and further obtain a first cell population.

Step 4: performing immunofluorescence staining of the blood cell surface marker and a tumor cell surface marker on the first cell population, and simultaneously performing nuclear staining, and further screening to obtain a first cell subpopulation and a second cell subpopulation; wherein the first cell subpopulation includes the first nucleated cells whose blood cell surface marker is negative and the tumor cell surface marker is positive; wherein the second cell subpopulation includes the second nucleated cells whose blood cell surface marker is negative and the tumor cell surface marker is negative.

Step 5: using a single cell analysis technique to analyze and measure the first cell subpopulation to further obtain a first nucleated cell information, and using the single cell analysis technique to analyze and measure the second cell subpopulation to further obtain a second nucleated cell information.

Step 6: inputting one of the first nucleated cell information and the second nucleated cell information or a combination of both to a cancer status analysis module together with a cancer clinical detection data of the individual for analysis, and further perform a prediction of cancer status.

In particular, the blood cell surface marker is selected from any one or any combination of the group consisting of CD3, CD4, CD8, CD11b, CD11c, CD14, CD19, CD20, CD33, CD34, CD41, CD45, CD56, CD61, CD62, CD66b, CD68, CD123, CD146, and Gly A.

In particular, the tumor cell surface marker is selected from any one or any combination of the group consisting of epithelial cell adhesion molecule (EpCAM), cytokeratins (CKs), epidermal growth factor receptor (EGFR), CD44, CD24, vimentin, mucin 1 (Muc-1), E-cadherin, N-cadherin, Ras, human epidermal growth factor receptor 2 (Her2), and MET.

In particular, the cancer clinical detection data is selected from any one or any combination of the group consisting of tumor blood marker detection data, tumor imaging detection data, tumor nucleic acid detection data, physical data of the subject, and medical record data of the subject.

Another aspect of the present invention is to offer the use of previous cancer status prediction method for cancer screening, cancer diagnosis, evaluating cancer prognosis, tracking and monitoring cancer status, or estimating the effectiveness of cancer treatment.

In some embodiments, the cancer comprising liver cancer, lung cancer, colorectal cancer, breast cancer, nasopharyngeal cancer, prostate cancer, esophageal cancer, pancreas cancer, skin cancer, thyroid cancer, stomach cancer, kidney cancer, gallbladder cancer, ovarian cancer, cervical cancer, bone cancer, brain cancer, or head and neck cancer.

In some embodiments, the single cell analysis technology is selected from any one or any combination of the group consisting of immunofluorescence staining, flow cytometry, fluorescence microscopy, immunomagnetic bead technology, microfluidic chip system, optical tweezers technology, dielectrophoretic microfluidic biochip, and light-induced dielectrophoretic microfluidic biochip system.

In some embodiments, the cancer status analysis module is selected from any one or any combination of the group consisting of biostatistical analysis, big data analysis and machine learning analysis.

In some embodiments, the cancer status analysis module is based on analyzing the statistical indicators comprising sensitivity, specificity, positive predictive value (PPV), negative predictive value (NPV), accuracy, area under ROC curve (AUC), or Yonden index for further cancer state prediction when step 6 is performing.

In some embodiments, the cancer status is cancer/cancer-free status classification, early-stage/late-stage cancer status classification, metastatic/non-metastatic cancer status classification, cancer treatment effective/ineffective status classification, or cancer recurrence/non-recurrence status classification.

In some embodiments, the tumor blood marker detection data is obtained from the individual in clinical detection of at least one tumor blood marker; wherein the tumor blood marker is further selected from any one or any combination of the group consisting of alpha fetal protein (AFP), carcinoembryonic antigen (CEA), carbohydrate antigen 19-9 (CA19-9), cytokeratin fragment 21-1 (CYFRA21-1), squamous cell carcinoma antigen (SCC), prostate specific antigen (PSA), carbohydrate antigen 15-3 (CA15-3), carbohydrate antigen 125 (CA125), Epstein-Barr virus IgA (EBV IgA), carbohydrate antigen 27-29 (CA27-29), beta-2-microglobulin, beta-human chorionic gonadotropin (Beta-hCG), cluster of differentiation 177 (CD 177), cluster of differentiation 20 (CD 20), chromogranin A (CgA), human epididymis secretory protein 4 (HE 4), lactate dehydrogenase (LDH), thyroglobulin, neuron-specific enolase (NSE), nuclear matrix protein 22, programmed death ligand 1 (PD-L1), prostatic acid phosphatase (PAP), tissue polypeptide antigen (TPA), tissue polypeptide specific antigen (TPS), tissue inhibitor of metalloproteinase-1 (TIMP-1), osteopontin (OPN), hepatocyte growth factor (HGF), myeloperoxidase (MPO), prolactin (PRL), and carbohydrate antigen 72-4 (CA72-4).

In some embodiments, the tumor imaging detection data is obtained from the individual clinically using a tumor imaging detection method; wherein the tumor imaging detection method is further selected from any one or any combination of the group consisting of X-ray imaging, computed tomography, positron emission tomography, ultrasonography, and nuclear magnetic resonance imaging.

In some embodiments, the tumor nucleic acid detection data is obtained from the individual in clinical detection of a nucleic acid marker; wherein the nucleic acid marker is further selected from any one or any combination of the group consisting of cancer-related genes AIP, BRCA2, CEP57, ERCC3, FANCD2, GATA2, MLH1, PHOX2B, SDHC, TP53, ALK, BRIP1, CHEK2, ERCC4, FANCE, GPC3, MSH2, PMS1, RB1, SDHD, TSC1, APC,BUB1B, CYLD, ERCC5, FANCF, HNF1A, MSH6, PMS2, RECOL4, SLX4, TSC2, ATM, CDC73, DDB2, EXT1, FANCG, HOXB13, MUTYH, PPM1D, RET, SMAD4, VHL, BAP1, CDH1, DICER1, EXTS, FANC1, HRAS, NBN, PRF1, RHBDF2, SIVIARCA4, WIT1, BARD1, CDK4, DIS3L2, EZH2, FANCL, KIT, NF1, PRKAR1A, RUXN1, SMARCB1, WRN, BLM, CDKN1C, EGFR, FANCA, FANCM, MAX, NF2, PTCH1, SBDS, STK11, XPA, BMPR1A, CDKN2A, EPCAM FANCB, FANCB, FH, MEN1, NSD1, PTEN, SDHAF2, SUFU, XPC, BRCA1, CEBPA, ERCC2, FANCC, FLCN, MET, PALB2, RAD51C, SDHB, TMEM127, ERBB2, NRAS, CTNNB1, PIK3CA, FBXW7, FGF2, FGFR2, KRAS, AKT1, PPP2R1A, GNAS, VIM, TIMP1, TIMP2, ICAM, MMP1, MMP9, MMP10, MM-13, MMP14, MMP15, MUC1, MDM2, NGFB, PDGFA, PDGFRA, TGFB1, TGFB2, CDKN2A, CDKN1B, PLAUR, CAV1, MGEA5, VEGF, CD44, CXCL14, ABCB1, ABCC1, ABCC2, ABCC3, ABCC5, CYP1A1, CYP1A2, CYP2B6, CYP2C9, CYP2D6, CYP2E1, CYP3A4, CYP3A5, CD274, ESR1, ESR2, MYC; and cancer-related microRNA let-7, microRNA-9 (miR-9), microRNA-10b (miR-10b), microRNA-17-5p (miR-17-5p), microRNA-18 (miR-18), microRNA-21 (miR-21), microRNA-23a (miR-23a), microRNA-23b (miR-23b), microRNA-23 (miR-23), microRNA-26a (miR-26a), microRNA-26b (miR-26b), microRNA-27a (miR-27a), microRNA-27b (miR-27b) microRNA-29s (miR-29s), microRNA-30a (miR-30a), microRNA-30d (miR-30d), microRNA-31 (miR-31), microRNA-34s (miR-34s), microRNA-92 (miR-92), microRNA-96 (miR-96), microRNA-100 (miR-100), microRNA-103 (miR-103), microRNA-107 (miR-107), microRNA-122a (miR-122a), microRNA-122 (miR-122), microRNA-124a (miR-124a), microRNA-125a (miR-125a), microRNA-125b (miR-125b), microRNA-128 (miR-128), microRNA-130 (miR-130), microRNA-133b (miR-133b), microRNA-135b (miR-135b), microRNA-140 (miR-140), microRNA-141 (miR-141), microRNA-143 (miR-143), microRNA-144 (miR-144), microRNA-145 (miR-145), microRNA-146b (miR-146b), microRNA-149 (miR-149), microRNA-155 (miR-155), microRNA-181a (miR-181a), microRNA-181b (miR-181b), microRNA-181d (miR-181d), microRNA-183 (miR-183), microRNA-184 (miR-184), microRNA-192 (miR-192), microRNA-196a (miR-196a), microRNA-197 (miR-197), microRNA-199a (miR-199a), microRNA-200a (miR-200a), microRNA-200c (miR-200c), microRNA-204 (miR-204), microRNA-205 (miR-205), microRNA-211 (miR-211), microRNA-212 (miR-212), microRNA-217 (miR-217), microRNA-221 (miR-221), microRNA-222 (miR-222), microRNA-224 (miR-224), microRNA-301 (miR-301), microRNA-320 (miR-320), microRNA-342 (miR-342), microRNA-372 (miR-372), microRNA-373 (miR-373), microRNA-375 (miR-375), microRNA-376a (miR-376a).

In some embodiments, the physical data of the subject is selected from any one or any combination of the group consisting of height, weight, gender, age, waist circumference, blood pressure, electrocardiogram, hearing, color blindness, vision examination, and various systematic examination of the individual.

In some embodiments, medical record data of the subject is selected from any one or any combination of the group consisting of family medical history, personal medical history, imaging medical history, and medication records.

### BRIEF DESCRIPTION OF THE DRAWINGS

For easier to understanding the above disclosure and other objects, features, advantages, and embodiments of the present invention, the description of the drawings is as follows:

FIG. 1 demonstrates a prediction performance graph drawn according to an embodiment of the present invention;

FIG. 2 demonstrates a prediction performance graph drawn according to another embodiment of the present invention.

According to the common operating method, the various features and elements in the figure are not drawn to actual scale. The drawing method aims to present the specific features and elements related to the present invention in the best way. In addition, the same or similar element symbols are used to indicated similar elements and components in different drawings.

### DETAILED DESCRIPTION OF THE INVENTION

Unless otherwise stated, all technical and scientific terms in the present application, including the specification and claims, have definitions known to the persons who are skilled and have general knowledge in the fields of medicine, pharmacy, and molecular biology. The definitions of several terms used in this specification are described below, and these definitions take precedence over general understanding in this specification.

### Definitions.

In the present invention, when a numerical value is accompanied by the term “about”, it is intended to include a range of ±10% of the value. The range of values includes all the values between the two ends and the values at the two ends. The “about” of the range applies to the two ends of the range; therefore, for example, “about 20-30” means the one that includes “20±10%-30±10%”.

In the present invention, the term “circulating tumor cell (CTC)” means a tumor cell that detaches from the primary tumor site and enters the circulatory system (blood and lymphatic system) of the human body. In terms of cell identification, circulating tumor cells must meet three characteristics includes a nucleus, tumor surface markers, and without blood cell surface markers (that is, nucleated cells with positive tumor surface markers and negative blood cell surface markers) in order to be separated from the rest of the circulating blood cells in the human body.

In the present invention, the term “threshold” refers to a value that can be used as a basis for determining whether the test result is positive or negative. A good threshold setting can be reflected in the high diagnostic sensitivity and high diagnostic specificity of the test result. Herein, the sensitivity refers to the true positive rate (that is, the proportion of those diagnosed as positive by the test to those who were found to be positive individuals), and the specificity refers to the true negative rate (that is, the proportion of those diagnosed as negative by the test to those who were found to be negative individuals). Regarding the setting of the threshold, various statistical analysis methods are used and implemented by a method known per se. For example, the threshold can be set by ROC analysis (receiver operating characteristics analysis), which is generally used as a method for reviewing the effectiveness of a diagnostic test. ROC analysis is based on the changes of threshold values, etc., to generate a ROC curve graph with the sensitivity of each threshold value as the vertical axis and FPF (false positive fraction, that is, 1-specificity value) as the horizontal axis. In the ROC curve, the inspection system with no diagnostic ability appears as a straight line on the diagonal. As the diagnostic ability increases, the more it appears to the upper left and upper arcs. Based on the description, it can be understood that the ROC curve of independent variables with precise sensitivity and specificity will be close to the upper left corner, and the point with the shortest distance from the upper left corner can be set as the threshold. In addition, the area under the curve of ROC can be used for calculation the diagnostic ability. The larger the value and the closer to 1, the better the diagnostic ability is.

### Embodiment

### Analysis of the Disease Status of 73 Patients with Confirmed Colorectal Cancer and 71 Healthy Subjects

In the present embodiment, the experiment was approved by the Institutional Review Board of the Chang Gung Memorial Hospital. All blood sample donors obtained informed consent (approval number: 201900267B0), and all methods were performed in accordance with relevant guidelines of clinical trials.

### Participant Information:

Table 1 demonstrates the participant information sheet of the present embodiment. As shown in Table 1, the embodiment included the circulating tumor cell information (CD45neg EpCAMneg cell and CD45neg EpCAMpos cell) and routine cancer detection data (the blood tumor marker CEA value and the age and gender data of the subjects) of 73 patients with confirmed colorectal cancer. The 73 patients with colorectal cancer were classified according to TNM stage: 1 subject in stage 0 (accounting for 1.4%), 14 subjects in stage 1 (accounting for 19.2%), 12 subjects in stage 2 (accounting for 16.4%), and 32 subjects in stage 3 (accounting for 43.8%), and 14 subjects in stage 4 (accounting for 19.2%). Furthermore, the gender ratio of cancer patients is 31 women (42.5%) and 42 men (57.5%). The average age is 62.7±13.06 years.

TABLE 1

| | | Healthy subjects | | Confirmed patients | | |
|---|---|---|---|---|---|---|
| | | Numbers/ | %/±Standard | Numbers/ | %/±Standard | |
| Variable | Category | Average | deviation | Average | deviation | P value |
| Population | — | 71 | — | 73 | — | — |
| Gender | Female | 39 | 54.9% | 31 | 42.5% | 0.1346 |
| | Male | 32 | 45.1% | 42 | 57.5% | — |
| Average age | — | 43.38 | ±12.17 | 62.7 | ±13.06 | <0.001 |
| Age group | Age < 65 years | 66 | 93.0% | 39 | 53.4% | <0.001 |
| | Age >= 65 years | 5 | 7.0% | 34 | 46.6% | — |
| TNM stage | stage 0 | — | — | 1 | 1.4% | — |
| | stage 1 | — | — | 14 | 19.2% | — |
| | stage 2 | — | — | 12 | 16.4% | — |
| | stage 3 | — | — | 32 | 43.8% | — |
| | stage 4 | — | — | 14 | 19.2% | — |

### Blood Sample Processing and Analysis:

Using flow cytometry to carry out negative screening and identification of circulating tumor cells for subjects, the steps are as follows:

Step 1: 8 mL of whole blood sample providing from the subject reacts with red blood cell lysis buffer to lyse the blood cells in the sample, and remove the supernatant by centrifugation.

Step 2: Centrifuge at 100-200×g to remove platelets to obtain a processed sample.

Step 3: The processed sample is negatively screened by the immunocytes separation method. Screening out at least one blood cell whose blood cell surface is marked as positive, and further obtain a first cell population; wherein the blood cell surface marker is selected from any one or any combination of the group consisting of CD3, CD4, CD8, CD11b, CD11c, CD14, CD19, CD20, CD33, CD34, CD41, CD45, CD56, CD61, CD62, CD66b, CD68, CD123, CD146, and Gly A. In the present embodiment, the blood cell surface marker is CD45. More specifically, the blood cell surface marking in this embodiment is based on the operation procedure of the EasySep™ CD45 depletion kit (StemCell Technologies, Vancouver, BC, Canada) to remove CD45-positive leukocytes to obtain the first cell population from negative screening.

Step 4: performing immunofluorescence staining of the blood cell surface marker and a tumor cell surface marker on the first cell population, and simultaneously performing nuclear staining, and further screening to obtain a first cell subpopulation, a second cell subpopulation, and a leukocyte cell subpopulation; wherein the tumor cell surface marker is selected from any one or any combination of the group consisting of epithelial cell adhesion molecule (EpCAM), cytokeratins (CKs), epidermal growth factor receptor (EGFR), CD44, CD24, vimentin, mucin 1 (Muc-1), E-cadherin, N-cadherin, Ras, human epidermal growth factor receptor 2 (Her2), and MET. In the present embodiment, the surface marker of the tumor cell is EpCAM. More specifically, the first cell subpopulation includes the first nucleated cells whose blood cell surface marker is negative and the tumor cell surface marker is positive (In this embodiment, it is CD45negative/EpCAMpositive cells); wherein the second cell subpopulation includes the second nucleated cells whose blood cell surface marker is negative and the tumor cell surface marker is negative (In this embodiment, it is CD45negative/EpCAMnegative cells); wherein the leukocyte cell subpopulation includes the leukocyte whose leukocyte surface marker is positive (In this embodiment, it is CD45positive leukocyte).

Step 5: using a single cell analysis technique to analyze and measure the first cell subpopulation to further obtain a first nucleated cell information, and using the single cell analysis technique to analyze and measure the second cell subpopulation to further obtain a second nucleated cell information; wherein the single cell analysis technology is selected from any one or any combination of the group consisting of immunofluorescence staining, flow cytometry, fluorescence microscopy, immunomagnetic bead technology, microfluidic chip system, optical tweezers technology, dielectrophoretic microfluidic biochip, and light-induced dielectrophoretic microfluidic biochip system. In the present embodiment, the single cell analysis technology analyzes and count cells by flow cytometry.

### Cancer Status Analysis Module:

A cancer status analysis module is established. Inputting one of the first nucleated cell information and the second nucleated cell information or a combination of both to the cancer status analysis module together with a cancer clinical detection data of the individual for analysis, and further perform a prediction of cancer status

In view of the above, the cancer status analysis module is selected from any one or any combination of the group consisting of biostatistical analysis, big data analysis and machine learning analysis; wherein the cancer clinical detection data is selected from any one or any combination of the group consisting of tumor blood marker detection data, tumor imaging detection data, tumor nucleic acid detection data, physical data of the subject, and medical record data of the subject.

Specifically, the tumor blood marker detection data is obtained from the individual in clinical detection of at least one tumor blood marker; wherein the tumor blood marker is further selected from any one or any combination of the group consisting of alpha fetal protein (AFP), carcinoembryonic antigen (CEA), carbohydrate antigen 19-9 (CA19-9), cytokeratin fragment 21-1 (CYFRA21-1), squamous cell carcinoma antigen (SCC), prostate specific antigen (PSA), carbohydrate antigen 15-3 (CA15-3), carbohydrate antigen 125 (CA125), Epstein-Barr virus IgA (EBV IgA), carbohydrate antigen 27-29 (CA27-29), beta-2-microglobulin, beta-human chorionic gonadotropin (Beta-hCG), cluster of differentiation 177 (CD 177), cluster of differentiation 20 (CD 20), chromogranin A (CgA), human epididymis secretory protein 4 (HE 4), lactate dehydrogenase (LDH), thyroglobulin, neuron-specific enolase (NSE), nuclear matrix protein 22, programmed death ligand 1 (PD-L1), prostatic acid phosphatase (PAP), tissue polypeptide antigen (TPA), tissue polypeptide specific antigen (TPS), tissue inhibitor of metalloproteinase-1 (TIMP-1), osteopontin (OPN), hepatocyte growth factor (HGF), myeloperoxidase (MPO), prolactin (PRL), and carbohydrate antigen 72-4 (CA72-4). In this embodiment, CEA is selected as the tumor blood marker, and one or a combination of the first nucleated cell information and the second nucleated cell information is input to the cancer state analysis module for processing, and further perform the prediction of the cancer state.

In different embodiments, the tumor imaging detection data can be selected as the cancer clinical detection data and input into the cancer status analysis module; wherein the tumor imaging detection data is obtained from the individual clinically using a tumor imaging detection method; wherein the tumor imaging detection method is further selected from any one or any combination of the group consisting of X-ray imaging, computed tomography, positron emission tomography, ultrasonography, and nuclear magnetic resonance imaging.

In different embodiments, the tumor nucleic acid detection data can be selected as the cancer clinical detection data and input into the cancer status analysis module; wherein the tumor nucleic acid detection data is obtained from the individual in clinical detection of a nucleic acid marker; wherein the nucleic acid marker is further selected from any one or any combination of the group consisting of cancer-related genes AIP, BRCA2, CEP57, ERCC3, FANCD2, GATA2, MLH 1, PHOX2B, SDHC, TP53, ALK, BRIP 1, CHEK2, ERCC4, FANCE, GPC3, MSH2, PMS1, RB1, SDHD, TSC1, APC, BUB1B, CYLD, ERCC5, FANCF, HNF1A, MSH6, PMS2, RECOL4, SLX4, TSC2, ATM, CDC73, DDB2, EXT1, FANCG, HOXB13, MUTYH, PPM1D, RET, SMAD4, VHL, BAP1, CDH1, DICER1, EXTS, FANC1, HRAS, NBN, PRF1, RHBDF2, SMARCA4, WIT1, BARD1, CDK4, DIS3L2, EZH2, FANCL, KIT NF1, PRKAR1A, RUXN1, SMARCB1, WRN, BLM CDKN1C, EGFR, FANCA, FANCM, MAX NF2, PTCH1, SBDS, STK11, XPA, BMPR1A, CDKN2A, FANCB, FANCB, FH, MEN1, NSD1, PTEN, SDHAF2, SUFU, XPC, BRCA1, CEBPA, ERCC2, FANCC, FLCN, MET PALB2, RAD51C, SDHB, TMEM127, ERBB2, NRAS, CTNNB1, PIK3CA, FBXW7, FGF2, FGFR2, KRAS, AKT1, PPP2R1A, GNAS, VIM TIMP1, TIMP2, ICAM, MMP1, MMP9, MMP10, MM-13, MMP14, MMP15, MUC1, MDM2, NGFB, PDGFA, PDGFRA, TGFB1, TGFB2, CDKN2A, CDKN1B, PLAUR, CAV 1, MGEA5, VEGF, CD44, CXCL14, ABCB1, ABCC1, ABCC2, ABCC3, ABCC5, CYP1A1, CYP1A2, CYP2B6, CYP2C9, CYP2D6, CYP2E1, CYP3A4, CYP3A5, CD274, ESR1, ESR2, MYC; and cancer-related microRNA let-7, microRNA-9 (miR-9), microRNA-10b (miR-10b), microRNA-17-5p (miR-17-5p), microRNA-18 (miR-18), microRNA-21 (miR-21), microRNA-23a (miR-23a), microRNA-23b (miR-23b), microRNA-23 (miR-23), microRNA-26a (miR-26a), microRNA-26b (miR-26b), microRNA-27a (miR-27a), microRNA-27b (miR-27b) microRNA-29s (miR-29s), microRNA-30a (miR-30a), microRNA-30d (miR-30d), microRNA-31 (miR-31), microRNA-34s (miR-34s), microRNA-92 (miR-92), microRNA-96 (miR-96), microRNA-100 (miR-100), microRNA-103 (miR-103), microRNA-107 (miR-107), microRNA-122a (miR-122a), microRNA-122 (miR-122), microRNA-124a (miR-124a), microRNA-125a (miR-125a), microRNA-125b (miR-125b), microRNA-128 (miR-128), microRNA-130 (miR-130), microRNA-133b (miR-133b), microRNA-135b (miR-135b), microRNA-140 (miR-140), microRNA-141 (miR-141), microRNA-143 (miR-143), microRNA-144 (miR-144), microRNA-145 (miR-145), microRNA-146b (miR-146b), microRNA-149 (miR-149), microRNA-155 (miR-155), microRNA-181 a (miR-181 a), microRNA-181b (miR-181b), microRNA-181d (miR-181d), microRNA-183 (miR-183), microRNA-184 (miR-184), microRNA-192 (miR-192), microRNA-196a (miR-196a), microRNA-197 (miR-197), microRNA-199a (miR-199a), microRNA-200a (miR-200a), microRNA-200c (miR-200c), microRNA-204 (miR-204), microRNA-205 (miR-205), microRNA-211 (miR-211), microRNA-212 (miR-212), microRNA-217 (miR-217), microRNA-221 (miR-221), microRNA-222 (miR-222), microRNA-224 (miR-224), microRNA-301 (miR-301), microRNA-320 (miR-320), microRNA-342 (miR-342), microRNA-372 (miR-372), microRNA-373 (miR-373), microRNA-375 (miR-375), microRNA-376a (miR-376a).

In different embodiments, the physical data of the subject can be selected as the cancer clinical detection data and input into the cancer state analysis module; wherein the physical data of the subject is selected from any one or any combination of the group consisting of height, weight, gender, age, waist circumference, blood pressure, electrocardiogram, hearing, color blindness, vision examination, and various systematic examination of the individual.

In different embodiments, the medical record data of the subject can be selected as the cancer clinical detection data and input into the cancer state analysis module; wherein medical record data of the subject is selected from any one or any combination of the group consisting of family medical history, personal medical history, imaging medical history, and medication records.

Furthermore, the cancer status analysis module is based on analyzing the statistical indicators comprising sensitivity, specificity, positive predictive value (PPV), negative predictive value (NPV), accuracy, area under ROC curve (AUC), or Yonden index for further cancer state prediction; wherein the cancer status is cancer/cancer-free status classification, early-stage/late-stage cancer status classification, metastatic/non-metastatic cancer status classification, cancer treatment effective/ineffective status classification, or cancer recurrence/non-recurrence status classification.

### The Performance of Cancer Status Prediction Data:

In the present embodiment, Table 2 demonstrates the performance of one or the combination of the first nucleated cell information and the second nucleated cell information in identifying cancer/cancer-free states.

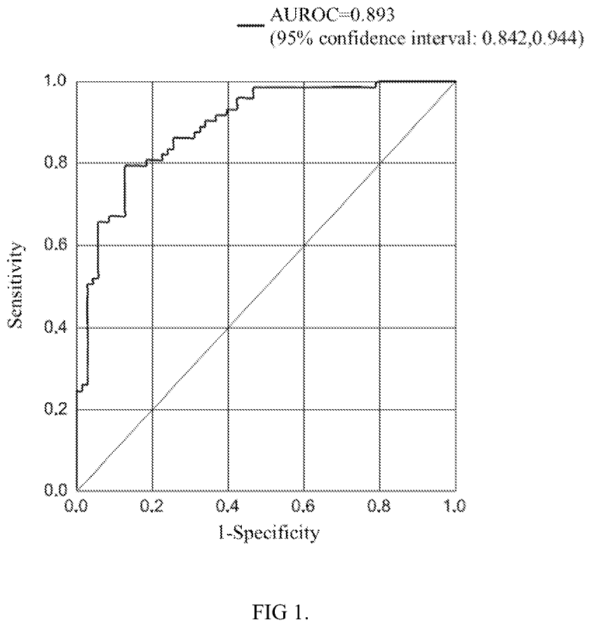

Specifically, the 73 patients with confirmed colorectal cancer were compared with the 71 healthy subjects. The statistical analysis for the first nucleated cell information (in this embodiment is CD45negative/EpCAMpositive cell) is based on the method of Counts, Continuous (that is, the impact of each additional first nucleated cell on the risk of diagnosed with cancer), which can obtain an AUC value of 0.882. After conversion by the Youden index, it has a sensitivity of 83.56% and a specificity of 81.69%. The statistical analysis for the second nucleated cell information (in this embodiment is CD45negative/EpCAMnegative cell) adopts 400 second nucleated cells/7m1 whole blood as the threshold, which can obtain an AUC value of 0.873. After conversion by the Youden index, it has a sensitivity of 79.45% and a specificity of 85.92%. Both of the above are better than conventional CEA detection performance (not shown in Table 2. AUC value is about 0.743, sensitivity is about 46.1%, and specificity is about 89.2%). Furthermore, according to FIG. 1, the first and second nucleated cell information are used together for analysis under the above conditions, an AUC value of 0.893 can be obtained.

It should be noted that the data presented in the table are adjusted statistically for gender and age.

TABLE 2

| | | Revised odds ratio | | AUROC |
|---|---|---|---|---|
| | | (95% confidence | | (95% confidence |
| Variable | Condition category | interval) | P value | interval) |
| The first | counts, continuous | 1.50 (1.12, 2.00) | 0.0065 | 0.882 (0.828, 0.937) |
| nucleated cell | count ≥ 2 v.s count < 2 | 1.99 (0.84, 4.75) | 0.1196 | 0.864 (0.803, 0.952) |
| (CD45negative/ | count ≥ 3 v.s count < 3 | 6.10 (1.77, 21.06) | 0.0042 | 0.875 (0.817, 0.933) |
| EpCAMpositive | count ≥ 4 v.s count < 4 | 5.12 (1.19, 22.11) | 0.0285 | 0.865 (0.804, 0.925) |
| cell) | | | | |
| The second | counts, continuous | 2.44 (1.06, 5.61) | 0.0354 | 0.868 (0.807, 0.928) |
| nucleated cell | ≥300 v.s <300 | 2.39 (1.02, 5.61) | 0.0449 | 0.867 (0.805, 0.926) |
| (CD45negative/ | ≥400 v.s <400 | 3.82 (1.54, 9.49) | 0.0039 | 0.873 (0.815, 0.931) |
| EpCAMnegative | ≥500 v.s <500 | 3.12 (1.22, 7.94) | 0.0172 | 0.869 (0.809, 0.929) |
| cell) | ≥600 v.s <600 | 2.92 (1.12, 7.62) | 0.0290 | 0.868 (0.807, 0.928) |
| The first | counts, continuous | 1.42 (1.07, 1.89) | 0.0164 | 0.893 (0.842, 0.944) |
| nucleated cell + | ≥400 v.s <400 | 2.84 (1.10, 7.35) | 0.0313 | |
| The second | | | | |
| nucleated cell | | | | |

In the present embodiment, Table 3 based on the first nucleated cell information (in this embodiment is CD45negative/EpCAMpositive cell), the second nucleated cell information (in this embodiment is CD45negative/EpCAMnegative cell), the cancer clinical detection data (in this embodiment is the cancer embryonic antigen CEA), or any combination of the above three for analyzing and predicting the status of early-stage/late-stage cancer and metastatic/non-metastatic cancer among the 73 patients with confirmed colorectal cancer. It should be noted that the distinction between early-stage/late-stage cancer in the embodiment is based on TNM classification, in which stage 0˜1 are early-stage cancer, and stage 2 or later are late-stage cancer. In addition, stage 0-3 is defined as non-metastatic cancer, and stage 4 is defined as metastatic cancer. On the other hand, the data presented in the table are statistically adjusted for gender and age.

TABLE 3

| | Condition category | | | | |
|---|---|---|---|---|---|
| | The first | The second | | early-stage/ | metastatic/ |
| | nucleated | nucleated | | late-stage | non-metastatic |
| Number of | cell | cell | | cancer AUROC | cancer AUROC |
| information | information | information | CEA | (95% confidence | (95% confidence |
| used | (continuous) | (≥500/7 ml) | (>5 ng/ml) | interval) | interval) |
| One | ✓ | | | 0.652 (0.508, 0.796) | 0.664 (0.497, 0.831) |
| | | ✓ | | 0.693 (0.549, 0.837) | 0.627 (0.478, 0.777) |
| | | | ✓ | 0.736 (0.604, 0.867) | 0.780 (0.645, 0.914) |
| Two | ✓ | ✓ | | 0.691 (0.546, 0.835) | 0.668 (0.504, 0.833) |
| | | ✓ | ✓ | 0.759 (0.631, 0.887) | 0.782 (0.650, 0.914) |
| | ✓ | | ✓ | 0.720 (0.585, 0.854) | 0.837 (0.740, 0.934) |
| Three | ✓ | ✓ | ✓ | 0.762 (0.635, 0.889) | 0.826 (0.706, 0.945) |

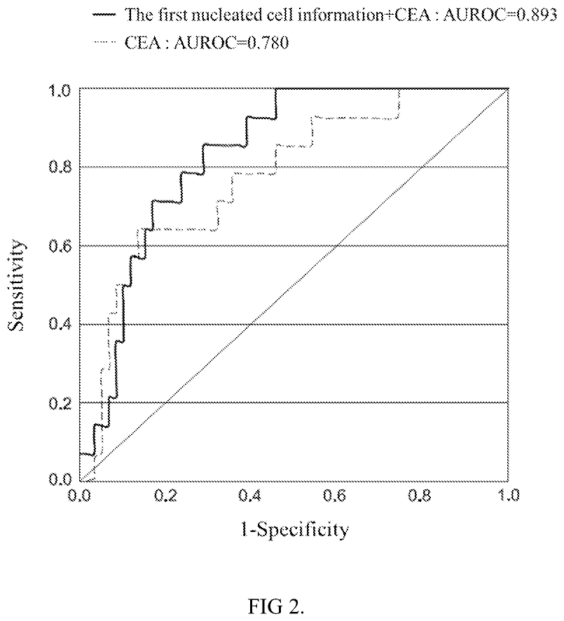

Refer to Table 3 and FIG. 2. When the first nucleated cell information is counted by Counts, Continuous, and the CEA data is analyzed together with a concentration greater than 5 ng/ml as the threshold value, it has an AUC value of 0.837 in the classification of metastatic/non-metastatic cancer status. When the above two are further combined with 500 pieces of the second nucleated cells/7 ml whole blood as the threshold to analyze the information of the second nucleated cell, it has an AUC value of 0.762 in the classification of early-stage/late-stage cancer status. The prediction performance of the above two combinations respectively for early-stage/late-stage cancer and metastatic/non-metastatic cancer status classification is better than the result of analyzing the CEA data alone.

According to different embodiments, the cancer state prediction method disclosed in the present invention can also be applied to cancer states other than colorectal cancer. In particular, the cancer comprising liver cancer, lung cancer, colorectal cancer, breast cancer, nasopharyngeal cancer, prostate cancer, esophageal cancer, pancreas cancer, skin cancer, thyroid cancer, stomach cancer, kidney cancer, gallbladder cancer, ovarian cancer, cervical cancer, bone cancer, brain cancer, or head and neck cancer.

According to the above content, the cell information in the blood sample can be combined with the cancer clinical detection data by the cancer state prediction method disclosed in the present invention for analyzed together to classified and predicted cancer/cancer-free status classification, early-stage/late-stage cancer status classification, metastatic/non-metastatic cancer status classification, and other cancer status classification. Further, the cancer state prediction method of the present invention is used for cancer screening, cancer diagnosis, evaluating cancer prognosis, tracking and monitoring cancer status, or estimating the effectiveness of cancer treatment.

The descriptions presented in embodiments of the specification are only technical philosophy and characteristics of the present application that can be understood and practiced by a person skilled in the art; the embodiments which should not be taken as examples to limit claims thereinafter may be modified or changed by the person based on the disclosed embodiments in the present disclosure without departing from the spirit of claims.

What is claimed is:

1. A method for predicting cancer status, comprising the following steps:
step 1: providing a whole blood sample from an individual;
step 2: performing a process on the whole blood sample to remove plural erythrocytes and platelets to further obtain a processed sample;
step 3: screening the processed sample by screening out at least one blood cell whose blood cell surface is marked as positive, and further obtain a first cell population;
step 4: performing immunofluorescence staining of the blood cell surface marker and a tumor cell surface marker on the first cell population, and simultaneously performing nuclear staining, and further screening to obtain a first cell subpopulation and a second cell subpopulation; wherein the first cell subpopulation includes the first nucleated cells whose blood cell surface marker is negative and the tumor cell surface marker is positive;
wherein the second cell subpopulation includes the second nucleated cells whose blood cell surface marker is negative and the tumor cell surface marker is negative;
step 5: using a single cell analysis technique to analyze and measure the first cell subpopulation to further obtain a first nucleated cell information, and using the single cell analysis technique to analyze and measure the second cell subpopulation to further obtain a second nucleated cell information;
step 6: inputting one of the first nucleated cell information and the second nucleated cell information or a combination of both to a cancer status analysis module together with a cancer clinical detection data of the individual for analysis, and further perform a prediction of cancer status; wherein
the blood cell surface marker is selected from any one or any combination of the group consisting of CD3, CD4, CD8, CD11b, CD11c, CD14, CD19, CD20, CD33, CD34, CD41, CD45, CD56, CD61, CD62, CD66b, CD68, CD123, CD146, and Gly A; wherein
the tumor cell surface marker is selected from any one or any combination of the group consisting of epithelial cell adhesion molecule (EpCAM), cytokeratins (CKs), epidermal growth factor receptor (EGFR), CD44, CD24, vimentin, mucin 1 (Muc-1), E-cadherin, N-cadherin, Ras, human epidermal growth factor receptor 2 (Her2), and MET; wherein
the cancer clinical detection data is selected from any one or any combination of the group consisting of tumor blood marker detection data, tumor imaging detection data, tumor nucleic acid detection data, physical data of the subject, and medical record data of the subject.

2. The method for predicting cancer status according to claim 1, wherein the cancer comprising liver cancer, lung cancer, colorectal cancer, breast cancer, nasopharyngeal cancer, prostate cancer, esophageal cancer, pancreas cancer, skin cancer, thyroid cancer, stomach cancer, kidney cancer, gallbladder cancer, ovarian cancer, cervical cancer, bone cancer, brain cancer, or head and neck cancer.

3. The method for predicting cancer status according to claim 1, wherein the single cell analysis technology is selected from any one or any combination of the group consisting of immunofluorescence staining, flow cytometry, fluorescence microscopy, immunomagnetic bead technology, microfluidic chip system, optical tweezers technology, dielectrophoretic microfluidic biochip, and light-induced dielectrophoretic microfluidic biochip system.

4. The method for predicting cancer status according to claim 1, wherein the cancer status analysis module is selected from any one or any combination of the group consisting of biostatistical analysis, big data analysis and machine learning analysis.

5. The method for predicting cancer status according to claim 1, wherein the cancer status analysis module is based on analyzing the statistical indicators comprising sensitivity, specificity, positive predictive value (PPV), negative predictive value (NPV), accuracy, area under ROC curve (AUC), or Yonden index for further cancer state prediction when step 6 is performing.

6. The method for predicting cancer status according to claim 1, wherein the cancer status is cancer/cancer-free status classification, early-stage/late-stage cancer status classification, metastatic/non-metastatic cancer status classification, cancer treatment effective/ineffective status classification, or cancer recurrence/non-recurrence status classification.

7. The method for predicting cancer status according to claim 1, wherein the tumor blood marker detection data is obtained from the individual in clinical detection of at least one tumor blood marker; wherein the tumor blood marker is further selected from any one or any combination of the group consisting of alpha fetal protein (AFP), carcinoembryonic antigen (CEA), carbohydrate antigen 19-9 (CA19-9), cytokeratin fragment 21-1 (CYFRA21-1), squamous cell carcinoma antigen (SCC), prostate specific antigen (PSA), carbohydrate antigen 15-3 (CA15-3), carbohydrate antigen 125 (CA125), Epstein-Barr virus IgA (EBV IgA), carbohydrate antigen 27-29 (CA27-29), beta-2-microglobulin, beta-human chorionic gonadotropin (Beta-hCG), cluster of differentiation 177 (CD 177), cluster of differentiation 20 (CD 20), chromogranin A (CgA), human epididymis secretory protein 4 (HE 4), lactate dehydrogenase (LDH), thyroglobulin, neuron-specific enolase (NSE), nuclear matrix protein 22, programmed death ligand 1 (PD-L1), prostatic acid phosphatase (PAP), tissue polypeptide antigen (TPA), tissue polypeptide specific antigen (TPS), tissue inhibitor of metalloproteinase-1 (TIMP-1), osteopontin (OPN), hepatocyte growth factor (HGF), myeloperoxidase (MPO), prolactin (PRL), and carbohydrate antigen 72-4 (CA72-4).

8. The method for predicting cancer status according to claim 1, wherein the tumor imaging detection data is obtained from the individual clinically using a tumor imaging detection method; wherein the tumor imaging detection method is further selected from any one or any combination of the group consisting of X-ray imaging, computed tomography, positron emission tomography, ultrasonography, and nuclear magnetic resonance imaging.

9. The method for predicting cancer status according to claim 1, wherein the tumor nucleic acid detection data is obtained from the individual in clinical detection of a nucleic acid marker; wherein the nucleic acid marker is further selected from any one or any combination of the group consisting of cancer-related genes AIP, BRCA2, CEP57, ERCC3, FANCD2, GATA2, MLH1, PHOX2B, SDHC, TP53, AIX, BRIP1, CHEK2, ERCC4, FANCE, GPC3, MSH2, PMS1, RB1, SDHD, TSC1, APC, BUB1B, CYLD, ERCC5, FANCF, HNF1A, MSH6, PMS2, RECOL4, SLX4, TSC2, ATM, CDC73, DDB2, EXT1, FANCG, HOXB13, MUTYH, PPM1D, RET, SMAD4, VHL, BAP1, CDH1, DICER1, EXTS, FANC1, HRAS, NBN, PRF1, RHBDF2, SMARCA4, WIT1, BARD1, CDK4, DIS3L2, EZH2, FANCL, KIT, NF1, PRKAR1A, RUX1V1, SMARCB1, WRN, BLM, CDKN1C, EGFR, FANCA, FANCM, MAX NF2, PTCH1, SBDS, STK11, XPA, BMPR1A, CDKN2A, EPCAM, FANCB, FANCB, FH, MEN1, NSD1, PTEN, SDHAF2, SUFU, XPC, BRCA1, CEBPA, ERCC2, FANCC, FLCN, MET, PALB2, RAD51C, SDHB, TMEM127, ERBB2, NRAS, CTNNB1, PIK3CA, FBXW7, FGF2, FGFR2, KRAS, AKT1, PPP2R1A, GNAS, VIM, TIMP1, TIMP2, ICAM, MMP1, MMP9, MMP10, MM-13, MMP14, MMP15, MUC1, MDM 2, NGFB, PDGFA, PDGFRA, TGFB1, TGFB2, CDKN2A, CDKN1B, PLAUR, CAV1, MGEA5, VEGF, CD44, CXCL14, ABCB1, ABCC1, ABCC2, ABCC3, ABCC5, CYP1A1, CYP1A2, CYP2B6, CYP2C9, CYP2D6, CYP2E1, CYP3A4, CYP3A5, CD274, ESR1, ESR2, MYC; and cancer-related microRNA let-7, microRNA-9 (miR-9), microRNA-10b (miR-10b), microRNA-17-5p (miR-17-5p), microRNA-18 (miR-18), microRNA-21 (miR-21), microRNA-23a (miR-23a), microRNA-23b (miR-23b), microRNA-23 (miR-23), microRNA-26a (miR-26a), microRNA-26b (miR-26b), microRNA-27a (miR-27a), microRNA-27b (miR-27b) microRNA-29s (miR-29s), microRNA-30a (miR-30a), microRNA-30d (miR-30d), microRNA-31 (miR-31), microRNA-34s (miR-34s), microRNA-92 (miR-92), microRNA-96 (miR-96), microRNA-100 (miR-100), microRNA-103 (miR-103), microRNA-107 (miR-107), microRNA-122a (miR-122a), microRNA-122 (miR-122), microRNA-124a (miR-124a), microRNA-125a (miR-125a), microRNA-125b (miR-125b), microRNA-128 (miR-128), microRNA-130 (miR-130), microRNA-133b (miR-133b), microRNA-135b (miR-135b), microRNA-140 (miR-140), microRNA-141 (miR-141), microRNA-143 (miR-143), microRNA-144 (miR-144), microRNA-145 (miR-145), microRNA-146b (miR-146b), microRNA-149 (miR-149), microRNA-155 (miR-155), microRNA-181a (miR-181a), microRNA-181b (miR-181b), microRNA-181d (miR-181d), microRNA-183 (miR-183), microRNA-184 (miR-184), microRNA-192 (miR-192), microRNA-196a (miR-196a), microRNA-197 (miR-197), microRNA-199a (miR-199a), microRNA-200a (miR-200a), microRNA-200c (miR-200c), microRNA-204 (miR-204), microRNA-205 (miR-205), microRNA-211 (miR-211), microRNA-212 (miR-212), microRNA-217 (miR-217), microRNA-221 (miR-221), microRNA-222 (miR-222), microRNA-224 (miR-224), microRNA-301 (miR-301), microRNA-320 (miR-320), microRNA-342 (miR-342), microRNA-372 (miR-372), microRNA-373 (miR-373), microRNA-375 (miR-375), microRNA-376a (miR-376a).

10. The method for predicting cancer status according to claim 1, wherein the physical data of the subject is selected from any one or any combination of the group consisting of height, weight, gender, age, waist circumference, blood pressure, electrocardiogram, hearing, color blindness, vision examination, and various systematic examination of the individual.

11. The method for predicting cancer status according to claim 1, wherein medical record data of the subject is selected from any one or any combination of the group consisting of family medical history, personal medical history, imaging medical history, and medication records.

12. The method as claimed in claim 1 is used for cancer screening, cancer diagnosis, evaluating cancer prognosis, tracking and monitoring cancer status, or estimating the effectiveness of cancer treatment.

13. The use as claimed in claim 12, wherein the cancer comprising liver cancer, lung cancer, colorectal cancer, breast cancer, nasopharyngeal cancer, prostate cancer, esophageal cancer, pancreas cancer, skin cancer, thyroid cancer, stomach cancer, kidney cancer, gallbladder cancer, ovarian cancer, cervical cancer, bone cancer, brain cancer, or head and neck cancer.

14. The use as claimed in claim 12, wherein the single cell analysis technology is selected from any one or any combination of the group consisting of immunofluorescence staining, flow cytometry, fluorescence microscopy, immunomagnetic bead technology, microfluidic chip system, optical tweezers technology, dielectrophoretic microfluidic biochip, and light-induced dielectrophoretic microfluidic biochip system.

15. The use as claimed in claim 12, wherein the cancer status analysis module is selected from any one or any combination of the group consisting of biostatistical analysis, big data analysis and machine learning analysis.

16. The use as claimed in claim 12, wherein the cancer status analysis module is based on analyzing the statistical indicators comprising sensitivity, specificity, positive predictive value (PPV), negative predictive value (NPV), accuracy, area under ROC curve (AUC), or Yonden index for further cancer state prediction when step 6 is performing.

17. The use as claimed in claim 12, wherein the cancer status is cancer/cancer-free status classification, early-stage/late-stage cancer status classification, metastatic/non-metastatic cancer status classification, cancer treatment effective/ineffective status classification, or cancer recurrence/non-recurrence status classification.

18. The use as claimed in claim 12, wherein the tumor blood marker detection data is obtained from the individual in clinical detection of at least one tumor blood marker;
wherein the tumor blood marker is further selected from any one or any combination of the group consisting of alpha fetal protein (AFP), carcinoembryonic antigen (CEA), carbohydrate antigen 19-9 (CA19-9), cytokeratin fragment 21-1 (CYFRA21-1), squamous cell carcinoma antigen (SCC), prostate specific antigen (PSA), carbohydrate antigen 15-3 (CA15-3), carbohydrate antigen 125 (CA125), Epstein-Barr virus IgA (EBV IgA), carbohydrate antigen 27-29 (CA27-29), beta-2-microglobulin, beta-human chorionic gonadotropin (Beta-hCG), cluster of differentiation 177 (CD 177), cluster of differentiation 20 (CD 20), chromogranin A (CgA), human epididymis secretory protein 4 (HE 4), lactate dehydrogenase (LDH), thyroglobulin, neuron-specific enolase (NSE), nuclear matrix protein 22, programmed death ligand 1 (PD-L1), prostatic acid phosphatase (PAP), tissue polypeptide antigen (TPA), tissue polypeptide specific antigen (TPS), tissue inhibitor of metalloproteinase-1 (TIMP-1), osteopontin (OPN), hepatocyte growth factor (HGF), myeloperoxidase (MPO), prolactin (PRL), and carbohydrate antigen 72-4 (CA72-4).

19. The use as claimed in claim 12, wherein the tumor imaging detection data is obtained from the individual clinically using a tumor imaging detection method; wherein the tumor imaging detection method is further selected from any one or any combination of the group consisting of X-ray imaging, computed tomography, positron emission tomography, ultrasonography, and nuclear magnetic resonance imaging.

20. The use as claimed in claim 12, wherein the tumor nucleic acid detection data is obtained from the individual in clinical detection of a nucleic acid marker; wherein the nucleic acid marker is further selected from any one or any combination of the group consisting of cancer-related genes AIP, BRCA2, CEP57, ERCC3, FANCD2, GATA2, MLH1, PHOX2B, SDHC, TP53, ALK, BRIP1, CHEK2, ERCC4, FANCE, GPC3, MSH2, PMS1, RB1, SDHD, TSC1, APC, BUB1B, CYLD, ERCC5, FANCF, HNF1A, MSH6, PMS2, RECOL4, SLX4, TSC2, ATM, CDC73, DDB2, EXT1, FANCG, HOXB13, MUTYH, PPM1D, RET, SMAD4, VHL, BAP1, CDH1, DICER1, EXTS, FANC1, HRAS, NBN, PRF1, RHBDF2, SMARCA4, WIT1, BARD1, CDK4, DIS3L2, EZH2, FANCL, KIT, NF1, PRKAR1A, RUX1V1, SMARCB1, WRN, BIM CDKN1C, EGFR, FANCA, FANCM, MAX, NF2, PTCH1, SBDS, STK11, XPA, BMPR1A, CDKN2A, EPCAM FANCB, FANCB, FH, MEN1, NSD1, PTEN, SDHAF2, SUFU, XPC, BRCA1, CEBPA, ERCC2, FANCC, FLCN, MET, PALB2, RAD51C, SDHB, TMEM127, ERBB2, NRAS, CTNNB1, PIK3CA, FBXW7, FGF2, FGFR2, KRAS, AKT1, PPP2R1A, GNAS, VIM MVP1, TIMP2, ICAM MMP1, MMP9, MMP10, MM-13, MMP14, MMP15, MUC1, MDM 2, NGFB, PDGFA, PDGFRA, TGFB1, TGFB2, CDKN2A, CDKN1B, PLAUR, CAV 1, MGEA5, VEGF, CD44, CXCL14, ABCB1, ABCC1, ABCC2, ABCC3, ABCC5, CYP1A1, CYP1A2, CYP2B6, CYP2C9, CYP2D6, CYP2E1, CYP3A4, CYP3A5, CD274, ESR1, ESR2, MYC; and cancer-related microRNA let-7, microRNA-9 (miR-9), microRNA-10b (miR-10b), microRNA-17-5p (miR-17-5p), microRNA-18 (miR-18), microRNA-21 (miR-21), microRNA-23a (miR-23a), microRNA-23b (miR-23b), microRNA-23 (miR-23), microRNA-26a (miR-26a), microRNA-26b (miR-26b), microRNA-27a (miR-27a), microRNA-27b (miR-27b) microRNA-29s (miR-29s), microRNA-30a (miR-30a), microRNA-30d (miR-30d), microRNA-31 (miR-31), microRNA-34s (miR-34s), microRNA-92 (miR-92), microRNA-96 (miR-96), microRNA-100 (miR-100), microRNA-103 (miR-103), microRNA-107 (miR-107), microRNA-122a (miR-122a), microRNA-122 (miR-122), microRNA-124a (miR-124a), microRNA-125a (miR-125a), microRNA-125b (miR-125b), microRNA-128 (miR-128), microRNA-130 (miR-130), microRNA-133b (miR-133b), microRNA-135b (miR-135b), microRNA-140 (miR-140), microRNA-141 (miR-141), microRNA-143 (miR-143), microRNA-144 (miR-144), microRNA-145 (miR-145), microRNA-146b (miR-146b), microRNA-149 (miR-149), microRNA-155 (miR-155), microRNA-181a (miR-181a), microRNA-181b (miR-181b), microRNA-181d (miR-181d), microRNA-183 (miR-183), microRNA-184 (miR-184), microRNA-192 (miR-192), microRNA-196a (miR-196a), microRNA-197 (miR-197), microRNA-199a (miR-199a), microRNA-200a (miR-200a), microRNA-200c (miR-200c), microRNA-204 (miR-204), microRNA-205 (miR-205), microRNA-211 (miR-211), microRNA-212 (miR-212), microRNA-217 (miR-217), microRNA-221 (miR-221), microRNA-222 (miR-222), microRNA-224 (miR-224), microRNA-301 (miR-301), microRNA-320 (miR-320), microRNA-342 (miR-342), microRNA-372 (miR-372), microRNA-373 (miR-373), microRNA-375 (miR-375), microRNA-376a (miR-376a).

21. The use as claimed in claim 12, wherein the physical data of the subject is selected from any one or any combination of the group consisting of height, weight, gender, age, waist circumference, blood pressure, electrocardiogram, hearing, color blindness, vision examination, and various systematic examination of the individual.

22. The use as claimed in claim 12, wherein medical record data of the subject is selected from any one or any combination of the group consisting of family medical history, personal medical history, imaging medical history, and medication records.
